(19) United States
(12) Patent Application Publication
(10) Pub. No.: US 2016/0256706 A1
(43) Pub. Date: Sep. 8, 2016

(54) Wearable Apparatus for Low Level Light Therapy Employing Semiconductor Light Sources

(71) Applicant: FLEXLITE CORPORATION, Tucson, AZ (US)

(72) Inventor: James Harrison, (US)

(21) Appl. No.: 15/032,307

(22) PCT Filed: Nov. 4, 2014
(86) PCT No.: PCT/US14/63830

§ 371 Date: Apr. 26, 2016

Related U.S. Application Data
(60) Provisional application No. 61/900,476, filed on Nov. 6, 2013.

(51) Int. Cl. A61N 5/06; A61H 1/00; A61N 1/362; A61F 7/02; A61N 1/36

(52) U.S. Cl. CPC A61N 5/0613; A61F 7/02; A61N 1/36003; A61N 1/362; A61H 1/006; A61N 2005/0645; A61N 2005/0652; A61N 2005/067

(57) ABSTRACT

A wearable low-level light therapy system which includes a device body configured to be detachably affixed to a body of a user proximate to an area of treatment, at least one semiconductor light source attached to the device body and configured to emit at least one optical signal to the area of treatment, at least one circuit is positioned on the device body and is in communication with the semiconductor light source and configured to regulate the operation of the multiple emitters forming the semiconductor light source, and at least one external controller in wireless communication with at least one circuit.

### CROSS-REFERENCE TO RELATED APPLICATIONS

The present application claims priority to U.S. Provisional Patent Application Ser. No. 61/900,476, entitled “Wearable Apparatus for Low Level Light Therapy Employing Semiconductor Light Sources,” filed on Nov. 6, 2013, the contents of which is incorporated by reference in its entirety herein.

### BACKGROUND

Presently, laser light is employed in a number of therapeutic applications for the treatment of mammals. For example, low-level light therapy is commonly used for pain management, to reduce inflammation, and to stimulate photo-biological response to enhance physiological reactions. Typically, appliances and systems used in low-level light therapy applications employ semiconductor Light Emitting Diodes (LEDs) and/or edge-emitting semiconductor lasers to generate optical outputs at wavelengths in the visible and/or near infrared spectral regions.

Generally, low-level light therapy processes require the non-invasive application of light to the skin of the patient proximate to a treatment area at a sufficient energy and wavelength configured to generate the desired therapeutic response. Ideally, the wavelength and power of the light incident on skin of the patient is sufficient to initiate photo-stimulation while not resulting in dermal or sub-dermal ablation or undesirable heating of the tissue. Presently, low-level light therapy systems utilize a large treatment device which is either strapped to the patient or held by a healthcare provider proximate to the area of treatment. Typically, the patient is required to remain stationary during the treatment process, which may range from several minutes to hours.

While presently available low-level light therapy systems have proven somewhat useful in the past, a number of shortcomings have been identified. For example, presently available systems require the patent to remain substantially stationary and immobile during treatment procedures. As such, this inconvenience may result in the patient foregoing needed treatment. Moreover, requiring a human patient to remain stationary during treatment may pose a substantial inconvenience; however, requiring other mammals to remain stationary during treatment may prove difficult if not impossible without sedation or other means. Further, presently available systems tend to be large, expensive systems more adapted for use in professional healthcare facilities.

In light of the foregoing, there is on ongoing need for a less expensive low-level light therapy system adapted to be worn by the patient without requiring the patient to be immobile.

### SUMMARY

The present application discloses various embodiments of a low-level light therapy and recovery system configured to by worn by or otherwise affixed to the patient or user. In one embodiment, the low-level light therapy and recovery system is configured to be used be a patient or user to treat a physiological condition. In the alternative, the low-level light therapy and recovery system may be used by an athlete or trainer to enhance cellular, muscular, and/or skeletal recovery before or after physical exertion or exercise. Unlike prior art light therapy systems which require the user to remain substantially immobile during treatment, the present system permits the user receive beneficial light therapy treatments to enhance recovery and/or therapeutic effects while continuing daily activity, recuperative rest, and/or or physical exercise.

In one embodiment, the present application is directed to a wearable low-level light therapy system which includes a device body configured to be detachably affixed to a body of a user proximate to an area of treatment. At least one semiconductor light source attached to the device body. The light source is configured to emit at least one optical signal to the area of treatment. The semiconductor light source may comprise a single emitter or, in the alternative, an array of the multiple emitters. At least one circuit is positioned on the device body and is in communication with the semiconductor light source. The circuit is configured to regulate the operation of the multiple emitters forming the semiconductor light source. Finally, the wearable low-level light therapy includes at least one external controller in wireless communication with at least one circuit. During use, the external controller is configured to provide data to and receive data from at least one of the multiple emitters, semiconductor light source, and the circuit.

In an alternate embodiment, the present application is directed to a wearable low-level light therapy system which includes a device body configured to be detachably affixed to a body of a user proximate to an area of treatment. At least one semiconductor light source is attached to the device body and configured to emit at least one optical signal to the area of treatment. In one embodiment, the semiconductor light source comprised of an array of the multiple emitters wherein at least one emitter comprises a vertical cavity surface emitting laser (hereinafter VCSEL). Finally, the wearable low-level light therapy system includes at least one circuit positioned on the device body and in communication with the semiconductor light source. The circuit may be configured to regulate the operation of the multiple emitters forming the semiconductor light source.

Other features and advantages of the embodiments of the wearable low-level light therapy system as disclosed herein will become apparent from a consideration of the following detailed description.

### BRIEF DESCRIPTION OF THE DRAWINGS

Various embodiments of the wearable low-level light therapy system will be explained in more detail by way of the accompanying drawings, wherein:

FIG. 1 shows an elevated perspective view of an embodiment of wearable low-level light therapy system having a light source and a circuit positioned on a device body;

FIG. 2 shows a schematic of an embodiment of an illumination system having two circuits controlling emitters of a light source used in a wearable low-level light therapy system;

FIG. 3 shows an elevated perspective view of an embodiment of wearable low-level light therapy system wherein the illumination system is selectively attached to a device body;

FIG. 4 shows a planar perspective view of an embodiment of a skeletal brace incorporating one or more wearable low-level light therapy systems therein;

FIG. 5 shows an elevated perspective view of another embodiment of a brace incorporating one or more wearable low-level light therapy systems therein;

FIG. 6 shows an elevated perspective view of another embodiment of a brace incorporating one or more wearable low-level light therapy systems therein;

FIG. 7 shows an elevated perspective view of an embodiment of a garment incorporating one or more wearable low-level light therapy systems therein;

FIG. 8 shows an elevated perspective view of another embodiment of a garment incorporating one or more wearable low-level light therapy systems therein; and

FIG. 9 shows a cross-sectional view of an embodiment of a wearable low-level light therapy system during use.

### DETAILED DESCRIPTION

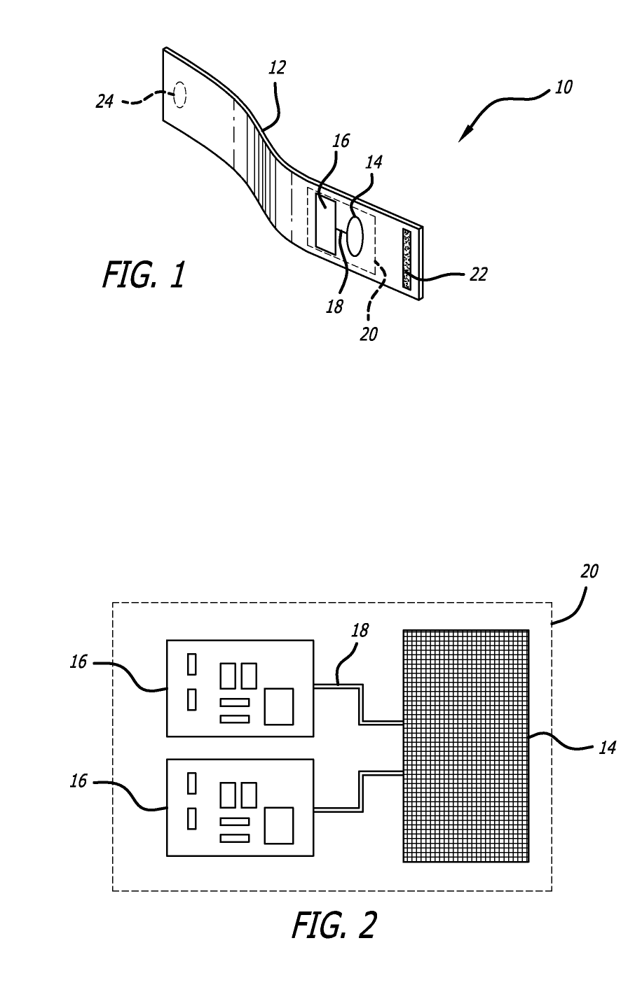

The low-level light therapy system disclosed herein utilizes at least one semiconductor light source configured to deliver at least one therapeutic optical signal to one or more areas of treatment. As shown in FIGS. 1-3, in one embodiment the low-level light therapy system 10 includes at least one device body 12. The device body 12 may be formed in any variety of shapes and sizes. Further, in one embodiment, the device body 12 is manufactured from at least one polymer material. Exemplary polymer materials include, without limitations, polyimide, neoprene, polyurethane, polyimide, nylon, and the like. Optionally, the device body 12 may be manufactured from a variety of materials, including, without limitations, polymers, natural fibers (e.g. wool, cotton, bamboo, etc.), silicon, elastomers, and the like. As such, the device body 12 may comprise a flexible, deformable body, a body configured to provide a compressive or expansive force, or, in the alternative, a rigid structure. Further, the device body 12 may include one or more light delivery devices integrated therein or attached thereto. For example, the device body 12 may include one or more fiber optic devices or waveguides integrated therein or coupled thereto.

Referring again to FIG. 1-3, at least one semiconductor light source 14 is coupled to device body 12. In one embodiment the light source 14 comprises at least one light emitting diode (hereinafter LED). Optionally, the LED may comprise super-luminescent and/or super-bright LED devices. In an alternate embodiment, the light source 14 comprises at least one laser diode. Exemplary laser diode configured for use with the present system include, without limitations, edge-emitting laser devices, VCSELs, and the like. Optionally, as shown in FIG. 2, the light source 14 may comprise an array of one or more emitters, such as LEDs or LED die, laser diodes or die, super-luminescent LEDs or die, or any combination thereof. For example, LEDs and VCSELs can be fabricated as compact, monolithic arrays of individual emitters to increase the total available power in operation as an ensemble surface-emitting light source. In such cases the individual emitters within an array can be electrically connected to facilitate electrical control of the ensemble as well as integration into flexible/stretchable/deformable electronic circuits. Multiple arrays could be similarly connected for ensemble operation and control. Optionally, the light source 14 need not include surface emitting devices. Optionally, the light source 14 may include one or more fiber optic lasers or fiber optic devices configured to deliver a therapeutic signal to various treatment areas. Further, the light source 14 may include any variety of light sources.

In some applications, semiconductor light sources are particularly well suited because of a combination of attributes including: high power-to-volume and high power-to-mass; low voltage and low power requirements; efficient conversion of electrical power to light; compatibility with flexible/stretchable electronic circuits and circuit assemblies; ability to operate at wavelengths of interest for low-level light therapy; reliability (e.g., in terms of expected hours of operation, durability); maturity of the technology and associated means of manufacturing; low cost per unit of light power (e.g., dollars per delivered Watt). In addition, semiconductor light sources offer high spatial coherence, facilitating illumination of remote target areas with minimal or no refractive optics. This is especially true of VCSEL versus edge emitting lasers. In addition, these sources have high spectral coherence, concentrating light energy at wavelengths of particular interest for specific low-level light therapy applications. Among semiconductor light sources, those based on III-V compounds including both Gallium and Arsenic are the most commonly used for low-level light therapy applications because of their high efficiency (conversion of electrical power to optical power), spectral compatibility with low-level light therapy applications and low cost.

Referring again to FIG. 1-3, in one embodiment, the light source 14 is configured to emit at last one therapeutic optical signal having a wavelength from about 400 nm to about 1500 nm. For example, in one embodiment, the light source 14 is configured to output at least one therapeutic optical signal having a wavelength from about 600 nm to about 1100 nm. In another embodiment, the light source 14 is configured to output at least one therapeutic optical signal having a wavelength from about 700 nm to about 1050 nm. In another embodiment, the light source 14 is configured to output at least one therapeutic optical signal having a wavelength from about 780 nm to about 1000 nm. In another application, the light source 14 is configured to output at least one therapeutic signal having a wavelength of about 700 nm to about 800 nm. In another embodiment, the light source 14 is configured to output at least one therapeutic signal having a wavelength of about 800 nm to about 900 nm. Optionally, a light source 14 may be configured to output multiple optical signals at a single wavelength or a narrow wavelength range. In another embodiment, the light source 14 may be configured to output any number of optical signals at different wavelengths. For example, the light source 14 may be configured to output a first therapeutic optical signal at a first wavelength and at least a second therapeutic optical signal at at least a second wavelength. Further, the light source 14 may be configured to output a continuous wave optical signal, a pulsed optical signal, and/or both. For example, in one embodiment at least one light source 14 is configured to emit at least one pulsed signal, the pulsed rate and/or pulse length of each pulse equal to a desired treatment protocol. Further, the light source 14 may include one or more optical elements positioned thereon or proximate thereto to condition or otherwise modify the therapeutic light emitted therefrom. For example, the light source 14 may include one or more filters, gratings, lenses, sensors, and the like positioned thereon or proximate thereto. For example, the light source 14 may include one or more optical metamaterials in optical communication therewith. Exemplary metamaterials include, without limitations, one or more ENZ (epilson near-zero) metamaterials thereby permitting the output of the light source 14 to be widely tunable over a desired range (e.g. all visible wavelengths). Optionally, the light source 14 may comprise a tunable light source configured to emit at least one optical signal within the range from about 400 nm to about 1500 nm.

As shown in FIG. 1-3, the low-level light therapy system 10 includes at least one circuit 16 in electrical communication with the light source 14. In one embodiment, the circuit 16 is configured to regulate the operation of and/or provide power to the light source 14. In another embodiment, the circuit 16 is configured to provide data to and receive data from the light source 14. Optionally, the circuit 16 may include one or more semiconductor devices, chips, sensors, controllers, processors, power supplies, batteries, energy sources, voltage regulators, current regulators, user interfaces, display devices, communication devices, user interfaces, wireless devices, MEMS devices, lab-on-a-chip systems, and the like. For example, in some embodiments, the circuit 16 includes one or more sensors configured to provide biological information and/or data received from the treatment area. Optionally, the biological information received from the treatment area maybe used to vary the treatment parameters such as the duration of the treatment, intensity of the illumination, pulse repetition rate, and the like. In addition, the circuit 16 may include one or more controllers configured to provide information, data, and/or one or more control signals to and receive information, data, and/or one or more control signals from one or more bio-medical sensors, controllers, and the like positioned external the body of the user and/or within the body of a user. For example, the circuit 16 may be in communication with at least one external controller (e.g. a smartphone, handheld device, computer, and the like) and at least one sensor or similar device positioned on or within the user. As such, the circuit 16 may act as a conduit configured to provide information to and receive information from the external control device and the sensor wirelessly and/or via a conduit. For example, the circuit 16 may be configured to provide and receive data from at least one of the light source 14, control pumps, drug delivery systems, pacemakers, and the like positioned on or within the body of a patient or user. For example, the circuit 16 may be configured to provide and receive data from multiple light source, additional circuits 16, and external controllers.

In addition, any number of additional sensors may be in communication with or included on the circuit 16. Exemplary additional sensors include, without limitation, flow sensors, oxygenation sensors, tissue temperature sensors, accelerometers, force meters, and the like. In one embodiment, the low-level light therapy system 10 includes one light source 14 and one circuit 16. Optionally, the low-level light therapy system 10 may include a single light source 14 in communication with multiple circuits 16. In another embodiment, the low-level light therapy system 10 includes multiple light sources 14 in communication with a single circuit 16. Further, the low-level light therapy system 10 may include multiple light sources 14 in communication with multiple circuits 16.

Further, the circuit 16 may include one or more integrated circuit devices, flexible circuits, and/or assemblies of integrated circuits and/or flexible circuits. Optionally, the circuit 16 may include one or more processors configured to be in communication at least one external controller (not shown). Exemplary external controllers include, for example, computers, handheld devices such as smart phones, tablet computers, computer networks, and the like. As such, at least one external processor may be configured to provide data to and/or receive data from at least one of the light source 14, circuit 16, and/or both via the circuit 16. Optionally, the circuit 16 and light source 14 may be combined to form an integrated and/or monolithic circuit and light source in a single unit.

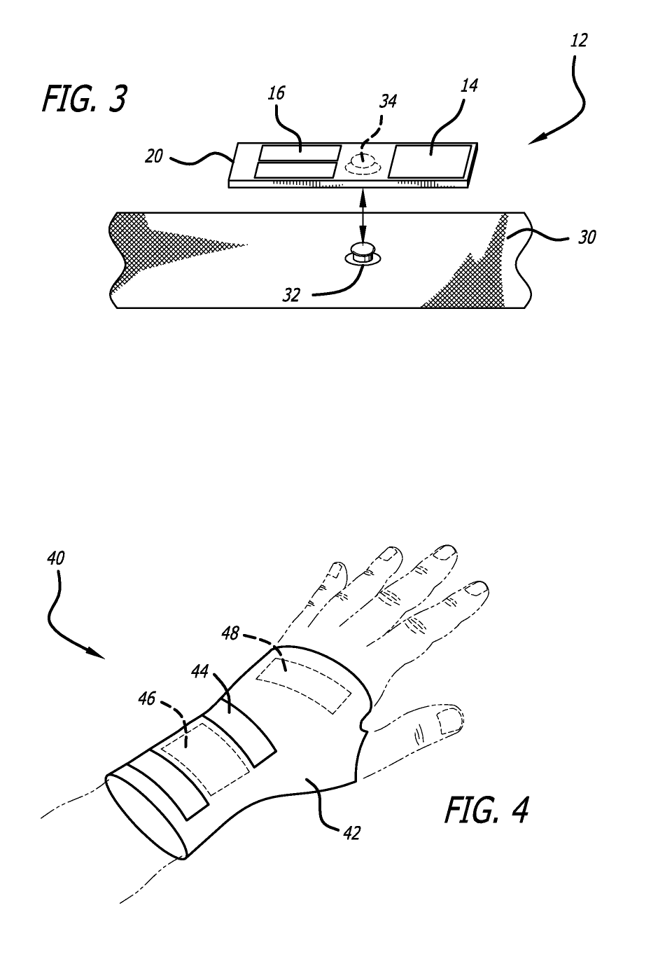

Optionally, as shown in FIGS. 1-3, the light source 14 and the circuit 16 may cooperatively form at least one illumination system body or area 20. In one embodiment, the light source 14 and circuit 16 are integral to the device body 12 of the low-level light therapy system 10. As such, the illumination system body 20 comprises an area containing the light source 14, circuit 16, and the at least one conduit 18 electrically coupling the light source 14 to the circuit 16. In another embodiment, at least one of the light source 14, circuit 16, or both may be detachably coupled to the device body 12. For example, in the embodiment shown in FIG. 3, the illumination system 20 including the light source 14 and circuit 14 are detachably coupled to device body 12. More specifically, the device body 12 may include at least one coupling area 30 formed thereon. In the illustrated embodiment, the coupling area 30 includes at least one coupling feature 32 configured to cooperatively attach to at least one coupling device 34 formed on or otherwise positioned on at least one of the light source 14, circuit 16, and/or illumination system body 20. As such, the illumination system 20 may be removed from the device body 12 in whole or in part, thereby permitting the device body 12 to be washed or otherwise treated (e.g. sterilization, cleaning, and the like) using conventional techniques without damaging the light source 14, circuit 16, conduits 18, and/or illumination system 20. Further, at least one of the light source 14, circuit 16, conduits 18, and/or illumination system 20 may include various housings or other devices to prevent environmental damage to the various components of the low-level light therapy system 10.

Referring again to FIGS. 1-3, in one embodiment the various components of the illumination system 20 may incorporate flex or stretchable electronic circuit technology. More specifically, flexible electronic circuits are by definition compatible with some degree of mechanical deformation. Commonly, flexible circuits are formed by mounting electronic components (e.g. the light source 14 and/or the circuit 16) on flexible substrates, with entire assemblies consisting of one or more (e.g., multi-layer) substrates. As such, at least one of the light source 14, circuit 16, conduit 18, and/or illumination system 20 may be mounted on at least one flexible substrate or may form a flexible electronic circuit. In the present application flexible electronic circuits are particularly useful when intended for deployment within, or as part of, wearable garments and/or accessories (e.g., bracelets). The flexibility of these circuits and the illumination system 20 can be enhanced both by the selection of substrate materials along with the design and selection of embedded components, electrical interconnects and mechanical structures forming the illumination system 20. As such, in one embodiment, the flexible circuits may be integrated into various garments, sleeves, braces, wraps, hats, and the like. Further, the effectively of the low-level light therapy system 10 may also be enhanced by optimizing the design of the low-level light therapy system 10 for use with of one or more garments, accessories, and/or attachment systems or mechanisms (e.g. tape, kinesiology tape, wraps, sleeves, braces, and the like). Optionally, the low-level light therapy system 10 may be configured for use with re-usable garments or disposable garments. For example, in one embodiment the low-level light therapy system 10 is configured for use with compressive garments, thereby providing therapeutic light therapy while simultaneously providing therapeutic compressive support. As such, in addition to providing compressive support, the compressive force of the compressive garment may securely position the low-level light therapy system 10 proximate to a treatment area on a user. In another embodiment, the low-level light therapy system 10 may be configured for use with disposable bandages, wraps, diapers, patches, and the like.

Optionally, one or more portable energy sources may be included within or otherwise coupled to the illumination system 20. For example, in one embodiment at least one power supply system is included within circuit 16 of the illumination system 20. Exemplary power supply systems include, for example, batteries. In one embodiment, the power supply system may be rechargeable. As such, the power supply system may be recharged by conventional means through a wired connection (e.g., utilizing a standardized connector such as a micro USB port) or via some form of wireless charging wherein the receiving antenna and conversion electronics are part of or in communication with the circuit 16. In fact, energy sourced from an external source separate from the low-level light therapy system could be transported wirelessly to directly supply some or all of the devices, components and sub-assemblies of the low-level light therapy system in lieu of batteries.

As shown in FIG. 1, the at least one attachment device 22 is coupled to, positioned on, or otherwise formed in the device body 12 of the low-level light therapy system 10. For example, in one embodiment, the attachment device 22 comprises hook and loop material thereby permitting the user to couple the low-level light therapy system 10 to the body of the user such that the light emitted from the light source 14 will be directed into the body of the user proximate to an area of interest or treatment area. Those skilled in the art will appreciate that any number and variety of attachment devices 22 may be used with the low-level light therapy system 10.

Referring again to FIG. 1, the low-level light therapy system 10 may further include one or more additional therapeutic systems or devices 24 coupled to the device body 12, light source 14, circuit 16, and/or illumination system 20. Exemplary additional therapeutic systems include, without limitations, muscle stimulations systems, compression systems, oxygen sensors, heart rate monitors, blood pressure monitors, thermometers, chillers/cooling elements, heaters, pumps, drug-delivery systems, pacemakers, diagnostic systems, and the like.

FIGS. 4-10 shows the various embodiments of the low-level light therapy system 10 disclosed herein incorporated into various braces and garments. For example, FIG. 4 shows an embodiment of a skeletal brace 40 configured to be applied to the wrist of a user to deliver therapeutic light to a treatment area. As shown, the brace 40 includes a brace body 42 having at least one attachment device 44 thereon. Further, the brace 40 includes at least one low-level light therapy system coupled thereto or included thereon. In the illustrated embodiment, a first low-level light therapy system 46 and a second low-level light therapy system 48 are positioned on the brace body 42 and configured to direct therapeutic light into the wrist of the user when worn by the user. Unlike prior art systems, the user of the brace 40 shown in FIG. 4, which includes the low-level light therapy system 46, 48, is not required to remain stationary. Rather, the user may preform substantially normal functions required in activities of daily life.

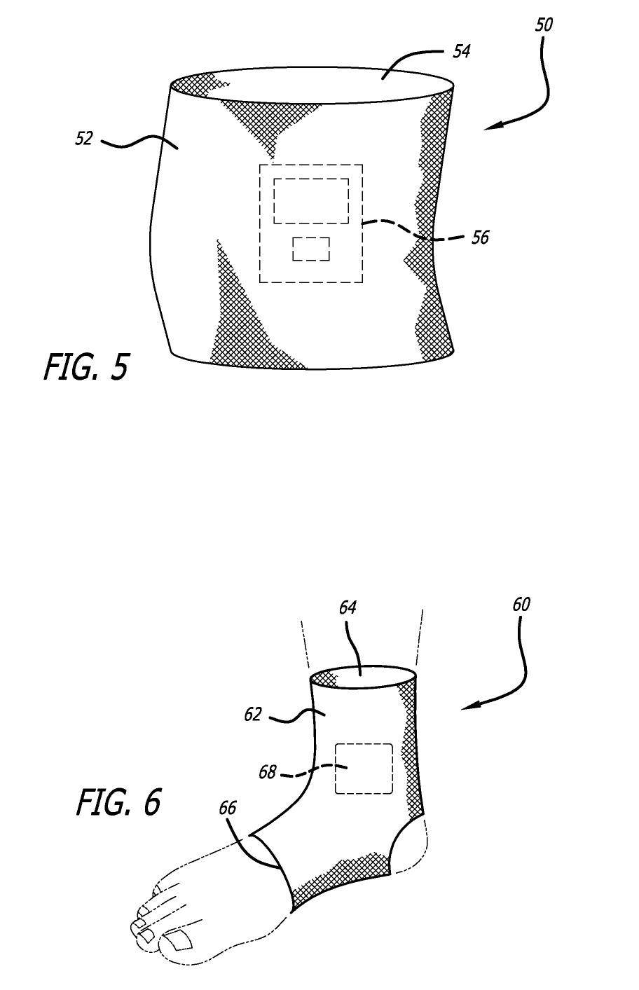

Similarly, FIG. 5 shows an embodiment of brace 50 configured to receive at least one body part therein. For example, the brace 50 shown in FIG. 5 may be configured for use on fingers, wrists, forearms, elbows, biceps, shoulders, triceps, hamstrings, quadriceps, knees, calves, toes, and the like. As shown, the brace 50 includes a brace body 52 defining at least one passage 54. Further, one or more low-level light therapy systems 56 may be coupled to or otherwise positioned on the brace 50 and configured to deliver therapeutic low-level light therapy to an area of interest. In the illustrated embodiment, the brace body 52 may be manufactured from spandex, polyurethane, neoprene, polyimide, or other compressive material and/or material combinations or blends configured to securely position and retain the low-level light therapy system 56 at a desired location.

FIG. 6 shows still another embodiment of the low-level light therapy system incorporated into a skeletal brace. As shown, the ankle brace 60 includes brace body 62 defining a first passage 64 sized to receive the low leg of the user and a second passage 66 sized to receive the foot of the user. Further, at least one low-level light therapy system 68 is coupled to or otherwise included on the brace 60 and configured to deliver therapeutic low-level light therapy to an area of interest. Like the previous embodiment, the brace body 62 may be manufactured from spandex, polyurethane, neoprene, polyimide, or other compressive material configured to securely position and retain the low-level light therapy system 68 at a desired location.

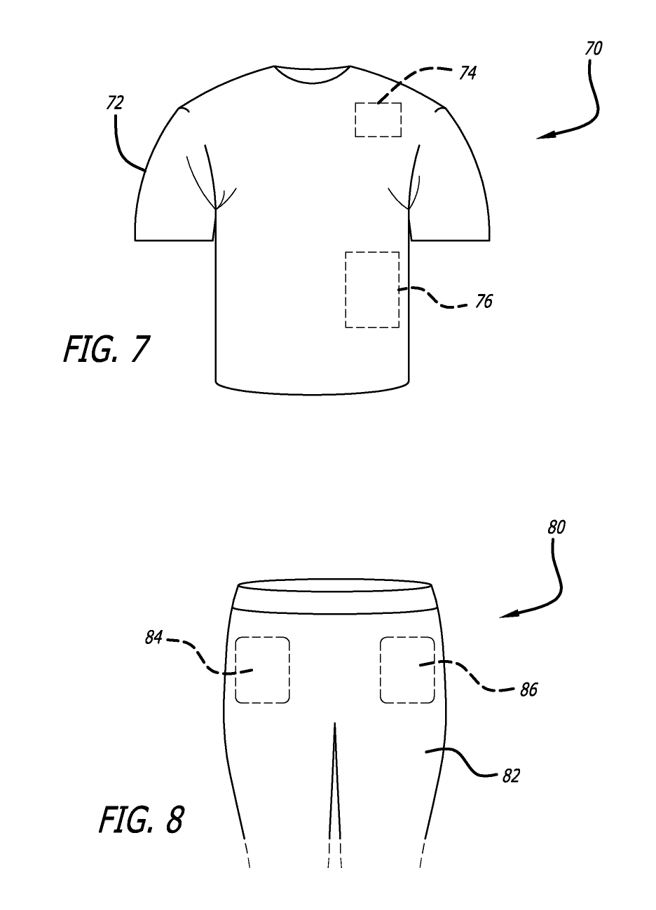

FIG. 7 shows another embodiment of the low-level light therapy system incorporated into a shirt and configured to deliver low-level light therapy to an area of interest located on the upper torso and/or shoulder of the user. As shown, the shirt 70 includes a shirt body 72 having at least one low-level light therapy system coupled thereto or included thereon. In the illustrated embodiment, a first one low-level light therapy system 74 and a second one low-level light therapy system 76 are detachably coupled to the shirt 70. During use, the user would couple the one low-level light therapy systems 74, 76 to the shirt using any variety of attachment devices (See FIG. 1, attachment device 22). Thereafter, the user would initiate the treatment process. For example, in one embodiment, the user would couple the one low-level light therapy system 74, 76 to at least one user control device (e.g. a handheld device, tablet computer, smartphone, etc.), select the treatment program and parameters from an application, programs or similar control software, and initiate and/or control the treatment process. Thereafter, while the treatment process is occurring, the user may continue his normal activities without being required to remain substantially stationary. In one embodiment, the shirt 70 is manufactured from spandex, polyurethane, neoprene, polyimide, or other compressive material configured to securely position and retain the low-level light therapy system 74, 76 at a desired location.

FIG. 8 shows another embodiment of the low-level light therapy system incorporated into a pants and/or shorts and configured to deliver low-level light therapy to an area of interest located on the lower torso of the user. As shown, the shorts 80 include a body 82 having at least one low-level light therapy system coupled thereto or included thereon. In the illustrated embodiment, a first one low-level light therapy system 84 and a second one low-level light therapy system 86 are detachably coupled to the shorts 80. During use, the user would couple the one low-level light therapy systems 84, 86 to the shorts using any variety of attachment devices (See FIG. 1, attachment device 22). Thereafter, the user would initiate the treatment process. For example, in one embodiment, the user would couple the one low-level light therapy system 84, 86 to at least one user control device (e.g. a handheld device, tablet computer, smartphone, etc.), select the treatment program and parameters from an application, programs or similar control software, and initiate the treatment process. Like the previous embodiment, while the treatment process is occurring, the user may continue his normal activities without being required to remain substantially stationary. In one embodiment, the shorts are manufactured from spandex, polyurethane, neoprene, polyimide, or other compressive material configured to securely position and retain the low-level light therapy system 84, 86 at a desired location.

As shown in FIGS. 4-8, low-level light therapy system disclosed herein may be attached to or otherwise incorporated into any number of garment, braces, and the like. Exemplary garments include, without limitations, shirts, pants shorts, socks, headbands, hats, caps, gloves, and the like. Similarly, the low-level light therapy system disclosed herein may be include within or coupled to skeletal splints, braces, sleeves, orthopedic braces (e.g. CTI-type devices), cervical collars, back braces, and the like. Further, the low-level light therapy system may be included within or coupled to various bandages, wraps, braces and the like used on mammals. As such, the low-level light therapy system disclosed herein may be easily configured to deliver a therapeutic treatment to various limbs, in whole or in part, joints, musculature, and the skeletal structure of a patient.

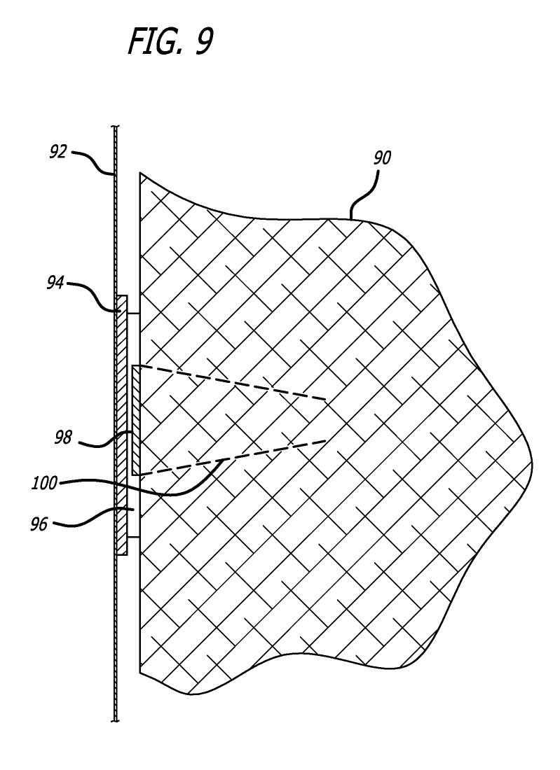

FIG. 9 shows an embodiment of a low-level light therapy system disclosed in the present application during use. As shown, the garment 92 (e.g. shirt) is worn by the user. In one embodiment, the garment 92 comprises a compression shirt configured to provide support compressive pressure to the musculature 90 of the user. At least one low-level light therapy system 94 is detachably coupled to the garment 92. As detailed above, the low-level light therapy system 94 includes at least one flexible circuit 96 in communication with at least one light source 98 configured to emit at least one optical signal 100 at a wavelength configured to stimulate a photo-biological response within the musculature 90 and/or other body constituent of the human and/or animal user. In the illustrated embodiment, the light source 98 is positioned immediately adjacent (e.g. in direct contact with or immediately proximate to) the musculature 90 and/or other body constituent of the human and/or animal user. In another embodiment, the light source 98 is positioned with a sleeve, protective garment, sterile pouch, and/or the like before being positioned proximate to the musculature 90 and/or other body constituent of the human and/or animal user. As stated above, the compressive force applied by the garment 92 is sufficient to maintain the low-level light therapy system 94 at a desired location during the treatment process.

The embodiments disclosed herein are illustrative of the principles of the invention. Other modifications may be employed which are within the scope of the invention. Accordingly, the devices disclosed in the present application are not limited to that precisely as shown and described herein.

1. A wearable low-level light therapy system, comprising:
a device body configured to be detachably affixed to a body of a user proximate to an area of treatment;
at least one semiconductor light source attached to the device body and configured to emit at least one optical signal to the area of treatment, the semiconductor light source comprised of an array of the multiple emitters;
at least one circuit positioned attached to the device body and in communication with the semiconductor light source, the circuit configured to regulate the operation of the multiple emitters forming the semiconductor light source; and
at least one external controller in wireless communication with the circuit and configured to provide data to and receive data from at least one of the multiple emitters, semiconductor light source, and the circuit.

2. The wearable low-level light therapy system of claim 1 wherein the device body is manufactured from a compressive material.

3. The wearable low-level light therapy system of claim 1 wherein the device body is manufactured from at least one deformable material.

4. The wearable low-level light therapy system of claim 1 wherein at least one emitter forming the semiconductor light source comprises at least one VCSEL.

5. The wearable low-level light therapy system of claim 1 wherein the semiconductor light source comprises one or more VCSELs and one or more LEDs.

6. The wearable low-level light therapy system of claim 1 wherein the semiconductor light source comprises one or more VCSELs and one or more super-luminescent LEDs.

7. The wearable low-level light therapy system of claim 1 wherein the semiconductor light source is configured to emit at least one continuous wave optical signal.

8. The wearable low-level light therapy system of claim 1 wherein the semiconductor light source is configured to emit at least one pulsed optical signal.

9. The wearable low-level light therapy system of claim 1 wherein the semiconductor light source is configured to emit an optical signal having a wavelength of about 400 nm to about 1500 nm.

10. The wearable low-level light therapy system of claim 1 wherein the semiconductor light source is configured to emit an optical signal having a wavelength of about 600 nm to about 1100 nm.

11. The wearable low-level light therapy system of claim 1 wherein the semiconductor light source is configured to emit an optical signal having a wavelength of about 700 nm to about 1050 nm.

12. The wearable low-level light therapy system of claim 1 wherein the semiconductor light source is configured to emit an optical signal having a wavelength of about 780 nm to about 1000 nm.

13. The wearable low-level light therapy system of claim 1 wherein the semiconductor light source is configured to emit an optical signal having a wavelength of about 700 nm to about 800 nm.

14. The wearable low-level light therapy system of claim 1 wherein the semiconductor light source is configured to emit an optical signal having a wavelength of about 800 nm to about 900 nm.

15. The wearable low-level light therapy system of claim 1 wherein the semiconductor light source is configured to emit a first optical signal at a first wavelength and at least a second optical signal at at least a second wavelength.

16. The wearable low-level light therapy system of claim 1 wherein the semiconductor light source is positioned immediately adjacent to the area of treatment.

17. The wearable low-level light therapy system of claim 1 wherein the semiconductor light source is positioned with at least one of a sleeve, protective garment, and sterile pouch before being positioned proximate to the area of treatment.

18. The wearable low-level light therapy system of claim 1 wherein the circuit includes at least one or more semiconductor devices, chips, sensors, controllers, processors, power supplies, batteries, energy sources, voltage regulators, current regulators, user interfaces, display devices, communication devices, user interfaces, wireless devices, MEMS devices, and lab-on-a-chip systems.

19. The wearable low-level light therapy system of claim 1 wherein the circuit includes at least one additional therapeutic system thereon.

20. The wearable low-level light therapy system of claim 1 wherein the additional therapeutic systems is selected from the group consisting of muscle stimulations systems, compression systems, biomedical sensors, oxygen sensors, heart rate monitors, blood pressure monitors, thermometers, chillers, cooling elements, heaters, pumps, drug-delivery systems, pacemakers, and diagnostic systems.

21. The wearable low-level light therapy system of claim 1 wherein the external controller comprises at least one computer.

22. The wearable low-level light therapy system of claim 1 wherein the external controller comprises at least one smartphone.

23. The wearable low-level light therapy system of claim 1 wherein the external controller comprises at least one hand-held device.

24. A wearable low-level light therapy system, comprising:
a device body configured to be detachably affixed to a body of a user proximate to an area of treatment;
at least one semiconductor light source attached to the device body and configured to emit at least one optical signal to the area of treatment, the semiconductor light source comprised of an array of the multiple emitters wherein at least one emitter comprises a VCSEL; and
at least one circuit positioned attached to the device body and in communication with the semiconductor light source, the circuit configured to regulate the operation of the multiple emitters forming the semiconductor light source.

25. The wearable low-level light therapy system of claim 24 wherein the device body is manufactured from a compressive material.

26. The wearable low-level light therapy system of claim 24 wherein the device body is manufactured from a deformable material.

27. The wearable low-level light therapy system of claim 24 wherein the semiconductor light source comprises one or more VCSELs and one or more LEDs.

28. The wearable low-level light therapy system of claim 24 wherein the semiconductor light source comprises one or more VCSELs and one or more super-luminescent LEDs.

29. The wearable low-level light therapy system of claim 24 wherein the semiconductor light source is configured to emit at least one continuous wave optical signal.

30. The wearable low-level light therapy system of claim 24 wherein the semiconductor light source is configured to emit at least one pulsed optical signal.

31. The wearable low-level light therapy system of claim 24 wherein the semiconductor light source is configured to emit an optical signal having a wavelength of about 400 nm to about 1500 nm.

32. The wearable low-level light therapy system of claim 24 wherein the semiconductor light source is configured to emit an optical signal having a wavelength of about 600 nm to about 1100 nm.

33. The wearable low-level light therapy system of claim 24 wherein the semiconductor light source is configured to emit an optical signal having a wavelength of about 700 nm to about 1050 nm.

34. The wearable low-level light therapy system of claim 24 wherein the semiconductor light source is configured to emit an optical signal having a wavelength of about 780 nm to about 1000 nm.

35. The wearable low-level light therapy system of claim 24 wherein the semiconductor light source is configured to emit an optical signal having a wavelength of about 700 nm to about 800 nm.

36. The wearable low-level light therapy system of claim 24 wherein the semiconductor light source is configured to emit an optical signal having a wavelength of about 800 nm to about 900 nm.

37. The wearable low-level light therapy system of claim 24 wherein the semiconductor light source is configured to emit a first optical signal at a first wavelength and at least a second optical signal at at least a second wavelength.

38. The wearable low-level light therapy system of claim 24 wherein the circuit includes at least one or more semiconductor devices, chips, sensors, controllers, processors, power supplies, batteries, energy sources, voltage regulators, current regulators, user interfaces, display devices, communication devices, user interfaces, wireless devices, MEMS devices, and lab-on-a-chip systems.

39. The wearable low-level light therapy system of claim 24 wherein the circuit includes at least one additional therapeutic system thereon.

40. The wearable low-level light therapy system of claim 24 wherein the additional therapeutic systems is selected from the group consisting of muscle stimulations systems, compression systems, biomedical sensors, oxygen sensors, heart rate monitors, blood pressure monitors, thermometers, chillers, cooling elements, heaters, pumps, drug-delivery systems, pacemakers, and diagnostic systems.

41. The wearable low-level light therapy system of claim 24 further comprising at least one external controller in communication with at least one of the semiconductor light source and the circuit, the external controller configured to provide data to and receive data from at least one of the semiconductor light source and the circuit.

42. The wearable low-level light therapy system of claim 41 wherein the external controller comprises at least one computer.

43. The wearable low-level light therapy system of claim 41 wherein the external controller comprises at least one smartphone.

44. The wearable low-level light therapy system of claim 41 wherein the external controller comprises at least one hand-held device.
